(19) United States
(12) Patent Application Publication
(10) Pub. No.: US 2024/0417672 A1
(43) Pub. Date: Dec. 19, 2024

(54) METHODS, SYSTEMS AND APPARATUS FOR ISOLATING EXOSOMES FROM A BIOLOGICAL SAMPLE

(71) Applicant: Vias Partners, LLC, Doylestown, PA (US)

(72) Inventors: Sanjay BATRA, Doylestown, PA (US); Elizabeth G. Cellucci, Doylestown, PA (US); Justin Joseph Batra, Doylestown, PA (US); Jaya Krishna Rose Batra, Doylestown, PA (US)

(21) Appl. No.: 18/820,029

(22) Filed: Aug. 29, 2024

Related U.S. Application Data
(63) Continuation-in-part of application No. 17/424,871, filed on Jul. 21, 2021.
(60) Provisional application No. 62/794,961, filed on Jan. 21, 2019.

(51) Int. Cl. C12N 1/02; C12N 1/38; C12N 5/078

(52) U.S. Cl. CPC C12N 1/02; C12N 1/38; C12N 5/0644

(57) ABSTRACT

Described herein are methods, systems and apparatus for separating components of a biological sample; and in particular, extracting and isolating exosomes. Methods of administering the exosomes are also described.

### BACKGROUND

Platelet-rich plasma (PRP) is generally understood to be a concentrate of platelets and plasma, that also contains growth factors, such as Platelet-Derived Growth Factor (PDGF); Transforming Growth Factor group (TGF); Epidermal Growth Factor (EGF); Vascular Endothelial Growth Factor (VEGF); Fibroblast Growth Factor (FGF); and Keratinocyte Growth Factor (KGF), which regulate the healing cascade by signaling surrounding cells to repair damaged tissue and regenerate new tissue.

Exosomes are membrane-bound extracellular vesicles (EVs) that are produced in the endosomal compartment of most eukaryotic cells. In multicellular organisms, exosomes and other EVs are found in biological fluids including saliva, blood, urine and cerebrospinal fluid. EVs have specialized functions in physiological processes, from coagulation and waste management to intercellular communication.

Platelets are filled with exosomes. Exosomes contain numerous anti-inflammatory growth factors, micro and messenger RNA. Platelets release these growth factors and RNA via exosomes to influence target cells through a paracrine function. Platelet derived exosomes (P-Es) have demonstrated similar effects in wound healing as PRP. Additionally, despite the small size of P-Es, they have numerous benefits, including their ability to be released locally, their ease of travel through the body, their low immunogenicity, and the ease with which they can be obtained.

Various methods and systems for preparing PRP are known; but for a variety of reasons, these methods and systems do not consistently provide efficient platelet capture or separation/isolation of exosomes from the platelets. For example, devices and systems utilizing a separator gel, tend to have issues with platelets adhering to the separator gel, resulting in suboptimal separation/isolation of exosomes from the platelets. As a result, the clinician is often left with a less than desirable number of platelets and/or exosomes available for administration to a patient.

Thus, there remains a need for simple, cost-effective, reliable and clinically useful methods for overcoming the aforementioned challenges; and that enrich platelet concentrations and increase the number of platelets and/or exosomes available for administration to a patient. Embodiments of the present invention are designed to meet these and other ends.

### SUMMARY

In some embodiments, the present invention is directed to methods for separating and/or isolating exosomes from a biological sample (e.g., from platelets). In some embodiments, the resultant exosomes have reduced red blood cells and leukocytes relative to whole blood to allow for use in the treatment of various conditions (e.g., orthopedics and cosmetics).

In some embodiments, the present invention is directed to methods for separating components of a biological sample, the method comprising: introducing a biological sample having a plurality of components to a tube comprising: a lumen; a proximal end; a distal end; an interior wall; and an exterior wall; applying a force to said tube for a time sufficient to separate said plurality of components; and agitating said tube at an angle (e.g. from about −15° to about) 90° effective to enrich the concentration of a component of the biological sample (e.g. platelets or exosomes).

Other embodiments provide compositions comprising a product produced by any one of the methods or systems described herein. While other embodiments provide methods of using a product produced by any one of the methods or systems described herein

Still further embodiments provide a system for separating and/or isolating exosomes from a biological sample comprising: a biological sample; a tube; a means for applying a centrifugal force to said tube; a means for agitating said tube; and optionally a means for activating a component of the biological sample to an extent sufficient to separate and/or isolate exosomes.

### BRIEF DESCRIPTION OF THE DRAWINGS

FIG. 1 depicts an exemplary tube of the present invention after centrifugation.

FIG. 2 depicts a first agitation angle.

FIG. 3 depicts a second agitation angle.

FIG. 4 depicts a third agitation angle according to certain embodiments of the present invention.

FIG. 5 depicts a fourth exemplary agitation angle according to certain embodiments of the present invention.

FIG. 6 depicts a fifth exemplary agitation angle according to certain embodiments of the present invention.

FIG. 7 illustrates the foam created by an exemplary embodiment of the present invention.

FIG. 8 illustrates the foam created by another exemplary embodiment of the present invention.

FIG. 9 illustrates the absence of foam when a comparative method is performed.

FIG. 10A and FIG. 10B depict data describing the isolation, purification and characterization of exosomes from human plasma samples.

FIG. 11 depicts the significant increase in exosomes created by an exemplary method of the present invention as compared to comparative methods with and without an activating agent.

### DETAILED DESCRIPTION

In some embodiments, the present invention provides a method for separating components of a biological sample, the method comprising: introducing a biological sample having a plurality of components to a tube comprising: a lumen; a proximal end; a distal end; an interior wall; and an exterior wall; applying a force to said tube for a time sufficient to separate said plurality of components; optionally wherein the creation of a thin layer of foam indicates that the components are separated to a sufficient extent.

In some embodiments, the tube is agitated at an angle of from about −15° to about 90°. In some embodiments, the force is a centrifugal force.

In some embodiments, the present invention is directed to methods for separating and/or isolating exosomes from the biological sample.

As part of the Instructions for Use (IFU), PRP systems typically call for a gentle inversion of the collection tube following centrifugation. The inversion allows for resuspension of the platelets in the sample of PRP.

In some embodiments, the present invention provides a method wherein a tube containing a separated biological sample is agitated along its long axis in a rapid manner at a rate of several times per second. In some embodiments, the method may be performed for a few seconds up to one minute. In other embodiments, the tube is agitated at an angle for about 5 seconds. In further embodiments, the tube is agitated for about 5 seconds and In certain embodiments, the agitation angle may be slightly negative (−15 degrees) to vertical (+90 degrees).

Without being bound by theory, the present inventors believe that the methods of the present invention create a washing (i.e., lavage of the surface of the separation barrier) that helps to release platelets that may be attached to, or adhere to the surface of the separation barrier thereby increasing the number of platelets available for resuspension and administration to a subject. The methods described herein are also able to separate/isolate exosomes from the platelets.

Some embodiments of the present invention provide a tube comprising a material selected from: glass; modified poly amide (MPA); polyethylene terephthalate (PET) and any other material which is inert to a biological sample. In some embodiments, the tube comprises a laminate structure wherein an exterior wall of the tube is made of a material different than the interior wall.

In some embodiments, the tube further comprises a stopper. In some embodiments, the stopper comprises a material inert to biological samples. In other embodiments, the stopper comprises a material that does not crumble. In certain embodiments, the stopper comprises butyl rubber or its halo derivative formulations. In further embodiments, the stopper has a hardness of from about forty (40) to sixty (60) Shore A. In other embodiments, the stopper has a hardness designed to provide stable vacuum for from about eighteen (18) to about twenty-four (24) months.

In some embodiments, the tube is capable of receiving biological samples of from about four (4) ml to about one hundred (100) ml. In other embodiments, the tube is designed to receive biological samples of from about eight (8) ml to about fifty (50) ml. Still further embodiments provide tubes designed to receive biological samples of from about ten (10) ml to about thirty (30) ml. Other embodiments provide tubes designed to receive biological samples of from about eleven (11) ml or about twenty-two (22) ml.

In some embodiments, the tube is selected from: a vacuum, tube, a non-vacuum tube, a plastic tube, a glass tube, a rigid tube, a non-rigid tube, a semi rigid tube and any combination thereof. In some embodiments, the terms “tube”, “collection tube”, “test tube”, and the like, may be used interchangeably.

In some embodiments, the tube further comprises a gel. In some embodiments, the gel comprises a thixotropic gel. In further embodiments, the gel comprises a polymer. In certain embodiments, the gel can be a homopolymer or a co-polymer comprising a combination of monomers. In some embodiments, the gel comprises a polyacrylate, polyolefin or polyester.

Still further embodiments provide a gel having a density at 25° C. of from about 1.03 g/cm³ to about 1.09 g/cm³. While other embodiments provide a gel having a density at 25° C. of from about 1.04 g/cm³ to about 1.07 g/cm³. In some embodiments, the gel has a density at 25° C. of from about 1.05 g/cm³.

In some embodiments, the gel has a viscosity at 30° C. of from about 1,000 to about 5,000 cps. In other embodiments, the gel has a viscosity at 30° C. of from about 1,000 to about 4,500 cps. In further embodiments, the gel has a viscosity at 30° C. of from about 1,000 to about 4,000 cps. While other embodiments utilize a gel having a viscosity at 30° C. of from about 1,000 to about 3,500 cps. Still further embodiments provide a gel having a viscosity at 30° C. of from about 1,000 to about 3,000 cps. In other embodiments, the gel has a viscosity at 30° C. of from about 1,500 to about 5,00 cps. In further embodiments, the gel has a viscosity at 30° C. of from about 2,000 to about 5,000 cps. While other embodiments utilize a gel having a viscosity at 30° C. of from about 2,500 to about 5,000 cps. Still further embodiments provide a gel having a viscosity at 30° C. of from about 3,000 to about 5,000 cps.

Yet other embodiments provide a separation barrier that does not comprise a gel, e.g. a solid float. In some embodiments, the float can take on a variety of shapes and may be constructed from a variety of materials. In certain embodiments, the float is comprised of a non-porous material and has a substantially smooth surface. In some embodiments, the separation barrier is selected from a gel; a solid float; and a combination thereof.

In some embodiments, the biological sample is autologous. In some embodiments, the biological sample comprises mammalian blood. In some embodiments, the mammalian blood comprises human blood. In some embodiments, the biological sample comprises whole blood.

Still further embodiments provide a biological sample comprising a first component comprising a plasma fraction and a second component comprising lymphocytes, monocytes and erythrocytes. In some embodiments, a centrifugal force is applied for a time sufficient to form a barrier between the first component and the second component. In other embodiments, a centrifugal force is applied for a time sufficient to form a barrier between the plasma fraction and the second component comprising lymphocytes, monocytes and erythrocytes.

In certain embodiments, the plasma fraction comprises platelets. In some embodiments, the plasma fraction comprises platelet rich plasma (PRP) and platelet poor plasma. In some embodiments, the plasma fraction comprises PRP and high-concentrated PRP. In some embodiments, the plasma fraction comprises PRP, high-concentrated PRP and ultra-high concentrated PRP.

Some embodiments further comprise the step of removing at least a portion of the first component. In some embodiments, from about twenty-five percent (25%) to about seventy-five percent (90%) of the first component is removed, optionally about thirty percent (30%) to about seventy percent (85%) of the first component is removed, about thirty-five percent (35%) to about sixty-five percent (80%) of the first component is removed, about forty percent (40%) to about sixty percent (75%) of the first component is removed, about forty-five percent (45%) to about fifty-five percent (70%) of the first component is removed, about forty-five percent (50%) to about fifty-five percent (90%) of the first component is removed, about fifty percent (50%), about sixty percent (60%), about seventy percent (70%), about eighty percent (80%), or about ninety percent (90%), of the first component is removed.

In some embodiments, the tube is agitated for a time sufficient to provide a plasma fraction having a straw color with a pinkish hue. In other embodiments, the tube is agitated for a time sufficient to provide a plasma fraction having a hue angle, h, in the CIELAB system of from 310 to 350 degrees. In further embodiments, the tube is agitated for a time sufficient to provide a plasma fraction having a hue angle, h, in the CIELAB system of from 310 to 345 degrees. In some embodiments, the tube is agitated for a time sufficient to provide a plasma fraction having a hue angle, h, in the CIELAB system of from 310 to 340 degrees. In still further embodiments, the tube is agitated for a time sufficient to provide a plasma fraction having a hue angle, h, in the CIELAB system of from 310 to 335 degrees. While in other embodiments, the tube is agitated for a time sufficient to provide a plasma fraction having a hue angle, h, in the CIELAB system of from 310 to 330 degrees. Still other embodiments provide methods wherein the tube is agitated for a time sufficient to provide a plasma fraction having a hue angle, h, in the CIELAB system of from 310 to 325 degrees. Yet other embodiments provide methods wherein the tube is agitated for a time sufficient to provide a plasma fraction having a hue angle, h, in the CIELAB system of from 310 to 320 degrees.

In some embodiments, the tube is agitated for a time sufficient to create a visually perceivable foam layer. In some embodiments, the foam layer is created on a surface of the plasma fraction. In some embodiments, the appearance of the foam layer correlates with the suspension of a clinically significant number of platelets in the plasma fraction. In other embodiments, the appearance of the foam is a signal that a clinically significant number of platelets are available for extraction and administration to a patient. In some embodiments, the visually perceivable foam layer comprises cellular debris.

In some embodiments, the terms “exosomes” and extracellular vesicles” are synonymous and may be used interchangeably.

In some embodiments, the foam layer has a thickness of from about one (1) millimeter to about ten (10) millimeters, optionally from about two (2) millimeters to about nine (9) millimeters, three (3) millimeters to about eight (8) millimeters, about four (4) millimeters to about seven (7) millimeters, or about 0.5 mm, about 1 mm, about 2 mm, about 3 mm, about 4 mm, about 5 mm, about 6 mm, about 7 mm, about 8 mm, about 9 mm, or about 10 mm. While in other embodiments, the foam layer has a density of from about 0.01 g/cm³ to about 0.25 g/cm³, optionally from about 0.05 g/cm³ to about 0.25 g/cm³, about 0.1 g/cm³ to about 0.25 g/cm³, about 0.15 g/cm³ to about 0.25 g/cm³, or about 0.2 g/cm³ to about 0.25 g/cm³.

In some embodiments, the tube is agitated for from about five (5) seconds to about sixty (60) seconds, optionally from about 5 seconds to about 50 seconds, about 5 seconds to about 45 seconds, about 5 seconds to about 40 seconds, about 5 seconds to about 35 seconds, about 5 seconds to about 30 seconds, about 5 seconds to about 25 seconds, about 5 seconds to about 20 seconds, about 5 seconds to about 15 seconds, or about 5 seconds to about 10 seconds.

In some embodiments, the agitation is stepwise. In some embodiments, the stepwise agitation comprises a plurality of five second intervals of agitation. In other embodiments, the stepwise agitation further comprises a break between five second intervals. In certain embodiments, the break is from about 0.1 seconds to about 5 seconds. In some embodiments, the five second intervals of agitation are repeated from about 2 to about 10 times, optionally 2, 3, 4, 5, 6, 7, 8, 9 or 10 times.

In some embodiments, the agitation is a rhythmic motion. In some embodiments, the agitation creates a longitudinal or transverse wave-like motion in the biological sample. In some embodiments, the agitation creates a mixed longitudinal and transverse wave-like motion in the biological sample.

In some embodiments, a centrifugal force of from about 500 g to about 5000 g is applied to said tube. In other embodiments, a centrifugal force of from about 750 g to about 5000 g is applied to said tube. While in other embodiments, a centrifugal force of from about 1000 g to about 5000 g is applied to said tube. In yet other embodiments, a centrifugal force of from about 1500 g to about 5000 g is applied to said tube. In some embodiments, a centrifugal force of from about 2000 g to about 5000 g is applied to said tube. In some embodiments, a centrifugal force of from about 2500 g to about 5000 g is applied to said tube. In some embodiments, a centrifugal force of from about 3000 g to about 5000 g is applied to said tube. In other embodiments, a centrifugal force of from about 3000 g to about 4000 g is applied to said tube. While in other embodiments, a centrifugal force of from about 1500 g to about 2500 g is applied to said tube.

In some embodiments, the centrifugal force creates a plasma-gel interface between a surface of the gel and a surface of the plasma fraction. In some embodiments, the plasma-gel interface comprises platelets. In certain embodiments, the platelets in the plasma-gel interface are releasably bound to a surface of the gel. In some embodiments, the agitation releases platelets from the plasma-gel interface. In some embodiments, the platelets released from the plasma-gel interface are suspended in the plasma fraction.

In some embodiments, the tube further comprises (or contains) an anticoagulant. In some embodiments, the anticoagulant is selected from: a citrate salt (e.g. buffered sodium citrate); an EDTA salt (potassium-ethylenediaminetetra-acid); citrate-theophylline-adenosine-dipyridamole (CTAD); hirudin, benzylsulfonyl-d-Arg-Pro-4-amidinobenzylamide (BAPA); citric/citrate dextrose (ACD); heparin; an iodo acetate salt; an oxalate salt; a fluoride salt; and a combination of two or more thereof. Certain embodiments of the present invention do involve the use of a tube comprising an anticoagulant. In such embodiments, the biological sample may have been pre-treated with anticoagulant or the biological sample does not need to be anticoagulated.

Other embodiments provide compositions comprising a product of any one of the methods or systems described herein. Still further embodiments provide for the use of a composition comprising a product of any one of the methods or systems described herein for treating or preventing alopecia, bed sores, wrinkles, pain, tendonitis, arthritis, acne, scarring, crow's feet, ligament sprains and tears, and/or skin lesions.

Still further embodiments provide systems for separating components of a biological sample comprising: a biological sample; a tube; a means for applying a centrifugal force to said tube (e.g. a centrifuge); and a means for agitating said tube. In some embodiments, the systems described herein further comprise a means for measuring color in a biological sample. In some embodiments, the means for measuring color in a biological sample is selected from a spectrophotometer and a densitometer.

In some embodiments, the centrifuge is selected from a fixed angle centrifuge and horizontal spin centrifuge, or a swinging bucket centrifuge.

In some embodiments, the means for agitating the tube is adapted to linearly agitate the tube. In some embodiments, the means for agitating the tube is a tube rocker.

Some embodiments of the present invention provide a system as described herein further comprising a platelet counter. While other embodiments further comprise a processor. In some embodiments, the processor is wirelessly coupled to the means for applying a centrifugal force; the means for agitating the tube; the means for measuring color in a biological sample; and the platelet counter. In some embodiments, the means for applying a centrifugal force; the means for agitating the tube; the means for measuring color in a biological sample; the platelet counter; and the processor are contained in a single apparatus.

As used herein, the term “available platelet count” (or “APC”) is intended to refer to the number of platelets that are readily accessible to the clinician for administration to a subject in need thereof.

In some embodiments, the methods and systems described herein increase the available platelet count (“APC”) by at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 200%, about 250%, about 300%, about 400%, or about 500%, versus the platelet count provided by a control system. In some embodiments, the control system is substantially similar system to those encompassed by the present invention, except for the absence of a means for agitating the tube; and/or a substantially similar system wherein the means for agitating the tube is only able to agitate the tube at an angle outside the scope of the ranges suitable for use in the claimed invention.

In some embodiments, the means for agitating the tube is adapted to agitate the tube at an angle of from about −10° to about 85°, about −5° to about 80°, about 0° to about 75°, about 5° to about 70°, about 10° to about 65°, about 15° to about 60°, about 15° to about 60°, about 20° to about 60°, about 25° to about 60°, about 25° to about 55°, about 25° to about 50°, about 30° to about 50°, about 35° to about 50°, about 40° to about 50°, about 5°, about 10°, about 15°, about 20°, about 25°, about 30°, about 35°, about 40°, about 45°, about 50°, about 55°, about 60°, about 65°, about 70°, about 75°, about 80°, about 85° or about 90°. As used herein, the term “agitation angle” and the like are intended to refer to the angle measured from horizontal.

In some embodiments, the methods and systems described herein provide an available platelet count (“APC”) of greater than about 375,000 platelets/microliter, about 400,000 platelets/microliter, about 425,000 platelets/microliter, about 450,000 platelets/microliter, about 475,000 platelets/microliter, about 500,000 platelets/microliter, about 525,000 platelets/microliter, about 550,000 platelets/microliter, about 575,000 platelets/microliter, about 600,000 platelets/microliter, about 625,000 platelets/microliter, about 650,000 platelets/microliter, about 675,000 platelets/microliter, about 700,000 platelets/microliter, about 725,000 platelets/microliter, about 750,000 platelets/microliter, about 775,000 platelets/microliter, about 800,000 platelets/microliter, about 825,000 platelets/microliter, about 850,000 platelets/microliter, about 875,000 platelets/microliter, about 900,000 platelets/microliter, about 925,000 platelets/microliter, about 950,000 platelets/microliter, or about 975,000 platelets/microliter.

In some embodiments, the present invention provides a method further comprising the step of activating the platelets to induce the release of exosomes. In some embodiments, the activating step comprises the application of a chemical activating agent. In some embodiments, the chemical activating agent is selected from: calcium chloride, calcium gluconate, thrombin, collagen, lipopolysaccharide, Ca²⁺ ionophores; and a combination of two or more thereof. In certain embodiments, the present invention provides a method that does not include the use of a chemical activating agent.

Other embodiments provide methods for: suspending platelets in a post-centrifuged biological sample; increasing APC in a biological sample; and/or enriching the platelet count in a biological sample, comprising: centrifuging a collection tube containing a biological sample and a thixotropic gel; and agitating the collection tube at an angle and rate effective to create a layer of foam on top of said biological sample.

For avoidance of doubt, at least a portion of any one of the methods described herein could be suitable for use in any one of the systems described herein.

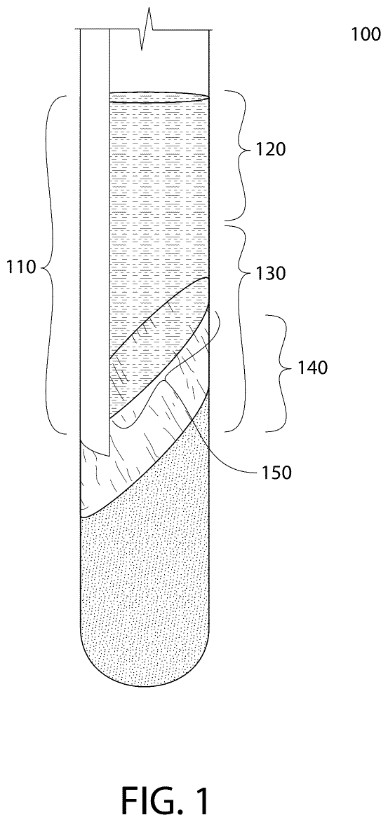

Referring first to FIG. 1, an exemplary tube (100) containing a biological sample post-centrifugation is depicted. As shown therein, the plasma fraction (110) comprises platelet poor plasma (120) and platelet rich plasma (130), wherein the platelet rich plasma (130) has a portion of ultra-high platelet concentration, sometimes referred to as ultra-high platelet rich plasma (140). Also depicted in FIG. 1 is the plasma-gel interface (150).

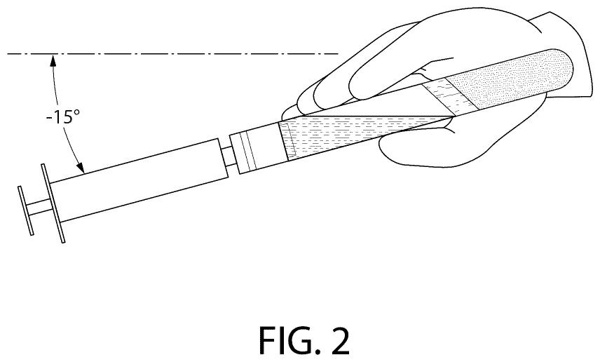

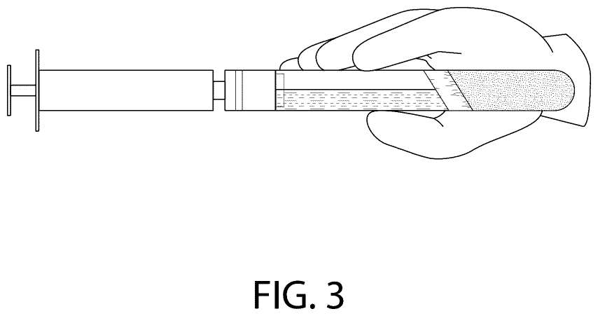

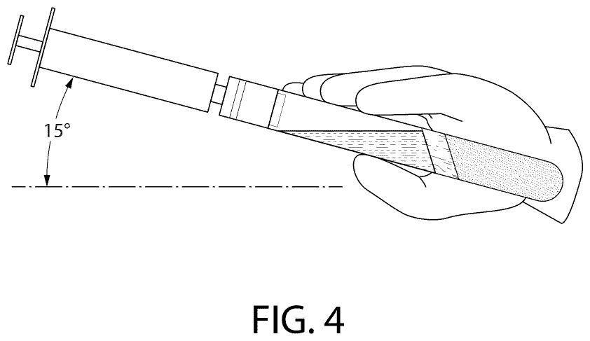

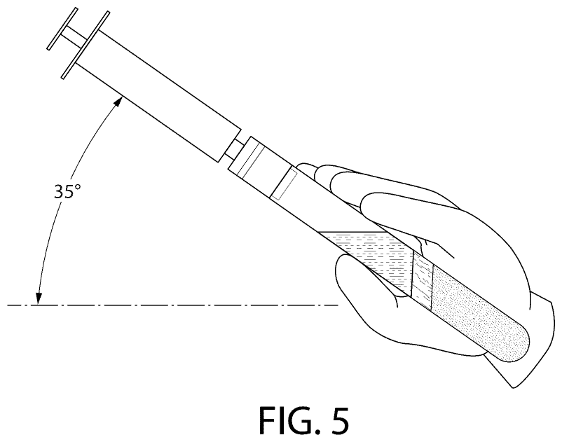

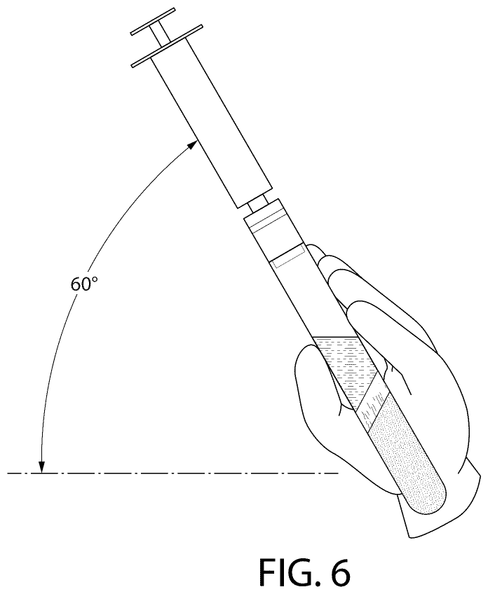

FIGS. 2-6 depict three exemplary agitation angles of the present invention and two comparative agitation angles. As discussed herein, the agitation angle is measured from the horizontal plane.

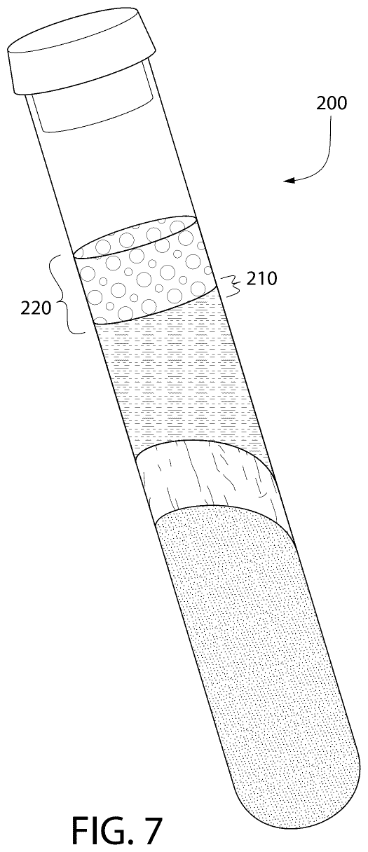

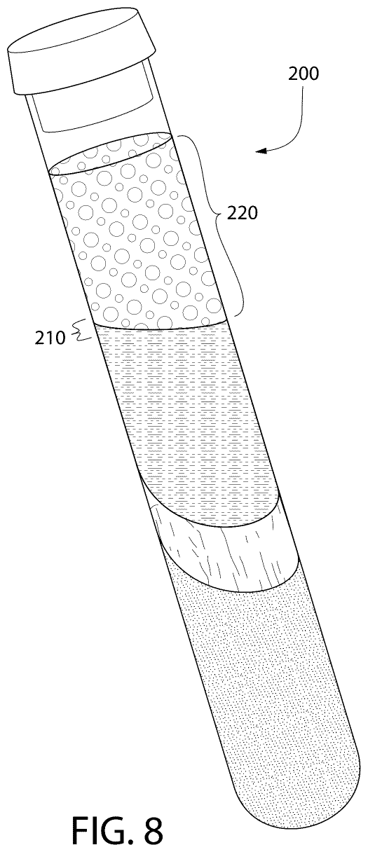

Referring next to FIGS. 7-8, a tube (200) containing a biological sample after centrifugation and agitation in accordance with certain embodiments of the present invention is depicted. FIGS. 7 and 8 also depict the layer of foam (220) that appears on the top surface of the plasma fraction (210).

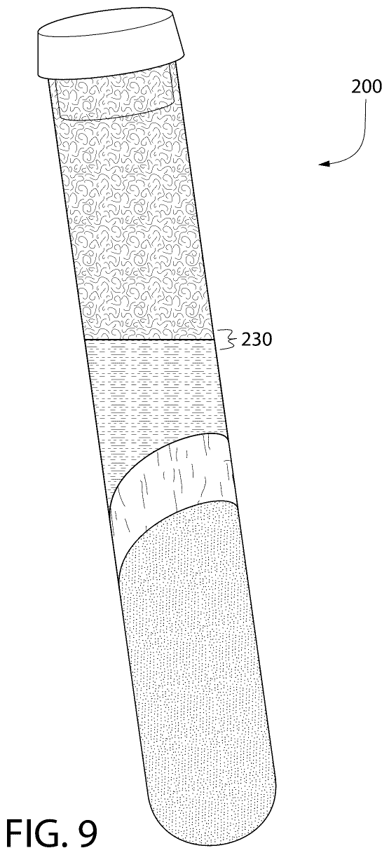

In contrast to FIGS. 7 and 8, tube (200) of FIG. 9 does not include a layer of foam on the top surface of the plasma fraction (230).

Further areas of applicability of the present invention will become apparent from the detailed description provided hereinafter. It should be understood that the detailed description and specific examples, while describing exemplary embodiments, are intended for purposes of illustration only and are not intended to limit the scope of the present invention.

### EXAMPLES

### Example 1

A series of experiments were conducted to compare exemplary methods of the present invention to current methods of preparing PRP; and to understand how certain features impact platelet suspension and capture. In particular, agitation angle, agitation time, color of the biological sample and the presence of foam were evaluated. Change in color and the presence of foam were evaluated at various time points during the experiments. As described in Table 1 (below), experiments conducted with exemplary agitation angles and agitation times of the present invention provided surprisingly increased platelet counts with minimal to no infiltration of unwanted cells from the biological sample (e.g. erythrocytes). The results of these experiments are described in Table 1 (below).

TABLE 1

| | Agitation | Agitation | | Platelet |
|---|---|---|---|---|
| | Angle | Time | | Count |
| Method | (°) | (seconds) | Color | (100K/μl) |
| Whole Blood | n/a | n/a | Deep red | 193 |
| Comp. Ex. 1* | n/a | n/a | Straw | 355 |
| Comp. Ex. 2 | 15 | 60 | Deep red | n/a |
| Comp Ex. 3 | 45 | 10 | Straw | 397 |
| Ex. 1 | 15 | 10 | Straw w/pink hue | 505 |
| Ex. 2 | 15 | 30 | Straw w/rose hue | 436 |
| Ex. 3 | 45 | 5 | Straw w/pink hue | 416 |
| Ex. 4 | 45 | 30 | Straw w/rose hue | 415 |
| Ex. 5 | 45 | 60 | Light rose | 480 |

Comp. Ex. 1 was a method performed in accordance with techniques known in the industry, wherein the
tube was gently inverted horizontally twenty times.

As illustrated by the data described in Table 1 (above) the exemplary methods of the present invention surprisingly increased platelet counts with acceptable levels of infiltrate. A foam layer was also observed with each of the exemplary methods of the present invention. Without being bound by theory, the present inventors believe that agitation angle, agitation time and agitation rate are critical to achieving clinically maximal platelet counts. In addition, the appearance of foam on top of the plasma fraction provides a signal to the clinician that the desired platelet concentration has been achieved, as it correlates with the increased platelet counts.

### Example 2

Additional experiments were conducted to further demonstrate the increased platelet counts provided by exemplary methods of the present invention. Five (5) samples from different donors were studied to evaluate the effect of the inventive methods at discrete time intervals ranging from five (5) seconds to one (1) minute. The impact of various agitation angles, ranging from −15° to 90° from horizontal, were also evaluated. The impact of both fixed angle and swing-bucket centrifuges was also evaluated. Platelet counts were performed using an automated Horiba ABX Micros 60 Hematology Analyzer (Horiba Instruments, Inc., Irvine CA).

The results of these experiments are described below in Tables 2 to 6. In each experiment 3 mL of platelet poor plasma (PPP) was removed before the platelets were counted in the PRP sample.
WB=Whole Blood
IFU=Instructions for Use
   IFU platelet counts refer to platelet counts obtained using techniques known in the industry, wherein the tube was gently inverted horizontally twenty times.

TABLE 2
WB Platelet Count = 202/IFU Platelet Count = 241

| | Setting | | Agitation Time (Seconds) | | | | | | | |
|---|---|---|---|---|---|---|---|---|---|---|
| Centrifuge | (speed × time) | Angle | 5 | 10 | 15 | 20 | 30 | 40 | 50 | 60 |
| Fixed | 3300 × 10 | −15 | 270 | 391 | 432 | 478 | 469 | n/a | — | — |
| Fixed | 3300 × 10 | 0 | 281 | 402 | 460 | 572 | 564 | 596 | 577 | — |
| Fixed | 3300 × 10 | 15 | 300 | 376 | 442 | 520 | 563 | 626 | 607 | — |
| Fixed | 3300 × 10 | 45 | 266 | 358 | 302 | 371 | n/a | — | — | — |
| Fixed | 3300 × 10 | 90 | 305 | 398 | n/a | — | — | — | — | — |

TABLE 3
WB Platelet Count = 335/IFU Platelet Count = 437

| | Setting | | Agitation Time (Seconds) | | | | | | | |
|---|---|---|---|---|---|---|---|---|---|---|
| Centrifuge | (speed × time) | Angle | 5 | 10 | 15 | 20 | 30 | 40 | 50 | 60 |
| Fixed | 3300 × 10 | −15 | 442 | 461 | 438 | 425 | n/a | — | — | — |
| Fixed | 3300 × 10 | 0 | 498 | 529 | 571 | 602 | 639 | n/a | — | — |
| Fixed | 3300 × 10 | 15 | 501 | 535 | 581 | 672 | 602 | 628 | 638 | 649 |
| Fixed | 3300 × 10 | 45 | 471 | 495 | 502 | 462 | n/a | — | — | — |
| Fixed | 3300 × 10 | 90 | 521 | n/a | — | — | — | — | — | — |

TABLE 4
WB Platelet Count = 263/IFU Platelet Count = 358

| | Setting | | Agitation Time (Seconds) | | | | | | | |
|---|---|---|---|---|---|---|---|---|---|---|
| Centrifuge | (speed × time) | Angle | 5 | 10 | 15 | 20 | 30 | 40 | 50 | 60 |
| Fixed | 3300 × 10 | −15 | 379 | 401 | n/a | — | — | — | — | — |
| Fixed | 3300 × 10 | 0 | 421 | 482 | 508 | 637 | 605 | n/a | — | — |
| Fixed | 3300 × 10 | 15 | 439 | 521 | 595 | 639 | 678 | 654 | 638 | — |
| Fixed | 3300 × 10 | 45 | 444 | 507 | 582 | 604 | n/a | — | — | — |
| Fixed | 3300 × 10 | 90 | 402 | n/a | — | — | — | — | — | — |

TABLE 5
WB Platelet Count = 321/IFU Platelet Count = 447

| | Setting | | Agitation Time (Seconds) | | | | | | | |
|---|---|---|---|---|---|---|---|---|---|---|
| Centrifuge | (speed × time) | Angle | 5 | 10 | 15 | 20 | 30 | 40 | 50 | 60 |
| Swing | 3300 × 10 | −15 | 487 | 521 | 591 | 667 | 582 | — | — | — |
| Swing | 3300 × 10 | 0 | 598 | 671 | 778 | 864 | 701 | 728 | — | — |
| Swing | 3300 × 10 | 15 | 608 | 788 | 901 | 853 | 846 | — | — | — |
| Swing | 3300 × 10 | 45 | 570 | 605 | 739 | 704 | 698 | — | — | — |
| Swing | 3300 × 10 | 90 | 683 | 721 | 629 | — | — | — | — | — |

TABLE 6
WB Platelet Count = 172/IFU Platelet Count = 304

| | Setting | | Agitation Time (Seconds) | | | | | | | |
|---|---|---|---|---|---|---|---|---|---|---|
| Centrifuge | (speed × time) | Angle | 5 | 10 | 15 | 20 | 30 | 40 | 50 | 60 |
| Swing | 3300 × 10 | −15 | 387 | 419 | 601 | 662 | 583 | 539 | — | — |
| Swing | 3300 × 10 | 0 | 408 | 488 | 573 | 701 | 745 | 705 | 728 | — |
| Swing | 3300 × 10 | 15 | 584 | 707 | 853 | 690 | 707 | — | — | — |
| Swing | 3300 × 10 | 45 | 551 | 674 | 779 | 871 | 720 | — | — | — |
| Swing | 3300 × 10 | 90 | 402 | 336 | — | — | — | — | — | — |

As illustrated by the data described in Tables 2 to 6 (above), exemplary methods of the present invention produce unexpected increases in platelet counts when compared to the platelet counts produced by conventional methods. These differences are not only numerically significant, but they also provide a clinically significant advance to the state of the art. Although the optimal time and angle may vary, the data unequivocally show that the agitation method, across the range of times and angles studied, increased platelet counts, thereby increasing the therapeutic dose of platelets that can be delivered to a subject.

### Example 3

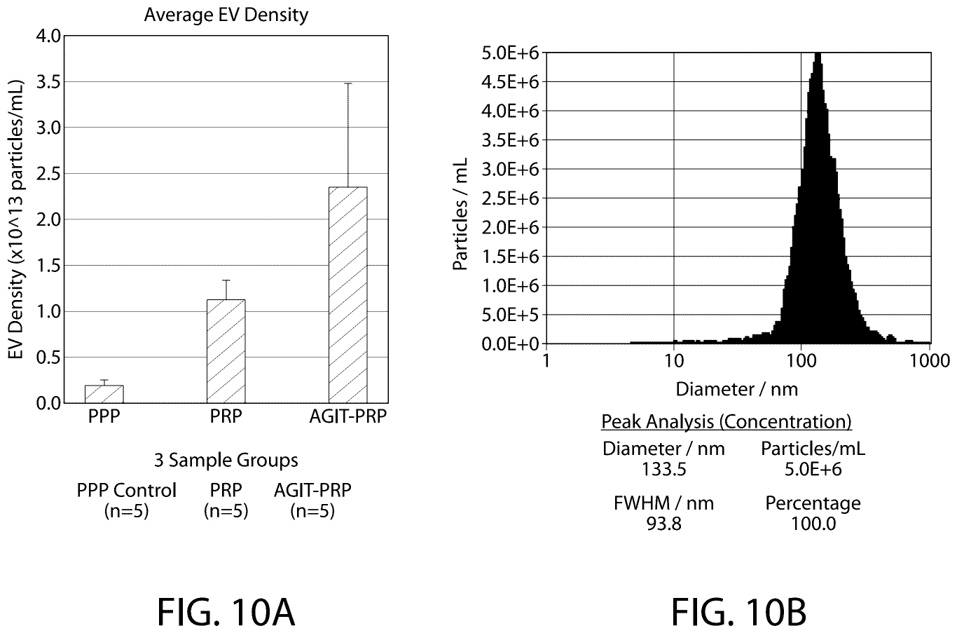

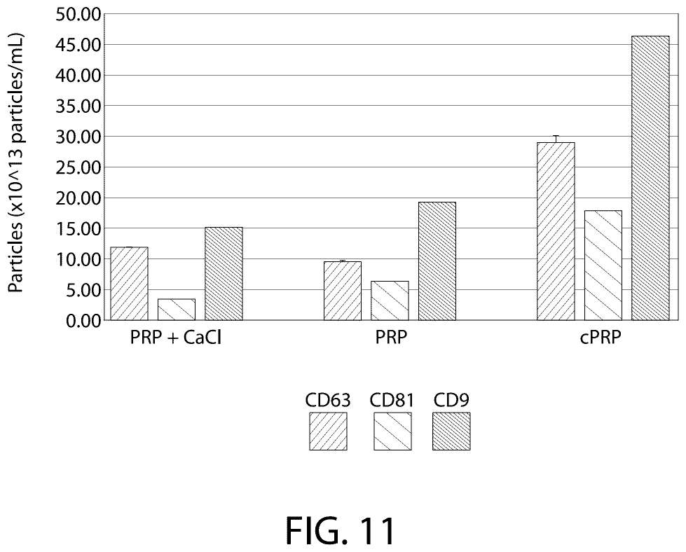

A series of experiments were conducted to compare exemplary methods of the present invention to current methods of separating/isolating exosomes; and to understand how certain features impact the quantity and density of exosomes in a biological sample. In particular, the experiments were run with and without a chemical activating agent. As described in FIG. 10A, FIG. 10B and FIG. 11, experiments conducted in accordance with exemplary methods of the present invention (e.g., without the use of a chemical activating agent) provided surprisingly increased exosome counts. The results are truly unexpected, in view of the general understanding that chemical activating agents are required to separate/isolate exosomes from platelets.

### Example 4

Approximately 75 ml total of human whole blood was obtained from each of five (5) donors following informed consent. Donors met the requirements of the American Association of Blood Banks (AABB), the FDA CBER and the Code of Federal Regulations: 21 CFR 606 and Title 45 Public Welfare-Department of Health and Human Services Part 46 Protection of Human Subjects. There were no specific exclusion criteria, other that the donor be healthy. There was no selection for age, sex or ethnicity. Donors were referenced only by assigned code numbers.

Platelet Rich Plasma (“PRP”) was prepared using two FDA cleared devices (Device 1 and Device 2). Peripheral whole blood was drawn according to manufacturer's instructions for use. 34-35 mL of whole blood was drawn into a syringe that had been prefilled 10% ACDA (3.5 mL). Device 1 was filled by vacuum to 30 mL whole blood, recognizing the gel tube already contained a small amount of the A-form of acid citrate dextrose (ACDA).

Samples were also obtained for baseline comparison of whole blood. The platelet concentrate samples were processed according to manufacturer instructions. Donor samples were disposed of in biohazardous waste following testing.

Device 1 was centrifuged in a Drucker Boost 2 Flex centrifuge for 10 minutes at 1600 G. Device 2 was centrifuged in an Elmi CM-7S centrifuge for 2 minutes at 2300 G.

The following end points were evaluated:
Platelet counts in PRP
PRP derived exosomes
PRP derived growth factors

### Platelet Counts

PRP samples (platelet fold increase, platelet percent capture, leukocytes and erythrocytes). All blood counts (WBC, HCT and Platelets) were performed using a Horiba Micros/ABX60 automated hematology analyzer.

Complete blood counts (CBCs) were performed using a 3-part differential hematology analyzer to quantify the platelets contained within the baseline sample, Device 1 and Device 2. The platelet concentration factor, which is the ratio of the concentration of platelets in the platelet concentrate product to the concentration of platelets in anticoagulated baseline sample, was determined for each device.

Complete blood counts (CBCs) were performed using a hematology analyzer to quantify the platelets contained within the baseline sample, JUVA PRP and REGEN BCT. The platelet yield, which is the ratio of the number of platelets in the platelet concentrate product to the number of platelets in the anticoagulated start sample, were determined for each device.

White Blood Cell, Hematocrit and Platelet Counts Complete blood counts (CBCs) were performed using a hematology analyzer for the baseline sample, Device 1 and Device 2. The White Blood Cell (WBC), Platelet (PLT) counts, as well as Hematocrit (HCT) were recorded for each sample.

### PRP Derived Exosomes: Concentration, Size and Size Distribution

Exosomes were isolated as follows. Initial centrifugation at 2000 G for 20 minutes at 4° C. the supernatant was pipetted off into a polycarbonate tube, ensuring not to disturb the pellet. Secondary centrifugation of the supernatant at 10,000 G for 30 minutes at 4° C. The supernatant was transferred to a fresh tube taking care to avoid collecting the pellet. A third centrifugation was performed at 100,000 G for 70 minutes at 4 C. Additional PBS was added to the sample if it was not three-quarters full (1×PBS without calcium, magnesium, and phenol red; Gibco, Thermo Fisher Scientific). The supernatant was then completely removed. The pellet was then resuspended in 1 mL of PBS, flushing up and down to mix thoroughly. Finally, the pellet was resuspended in 50-100 μL of PBS and frozen at −80° C., taking care to only have one freeze-thaw cycle to maintain sample integrity.

Isolated exosomes were quantified for concentration, particle size and particle size distribution using the Zetaview particle metrix, which employs nano particle tracking methodology. To determine EVs size and concentration, NTA was performed with the Zeta View PMX 110 NTA device (Particle Metrix GmbH, Inning am Ammersee, Germany) and ZetaView NTA software (8.04.02 SP2) for data analysis. The instrument was calibrated using a 100 nm polystyrene nanoparticles standard (Applied Microspheres BV, Leusden, The Netherlands, cat. no. 10100). The Brownian motion of each particle was visualized by a laser light scattering method, recorded by a camera and converted into size and concentration parameters using the Stokes-Einstein equation. Each EV sample was diluted with 1×PBS (1:1000; cat. no. D8537, Sigma-Aldrich) to ensure that the concentration of particles in each sample was optimal, and then counted in three cycles at 11 points in the NTA flow cell. The following settings were utilized: sensitivity 85, shutter speed 70, and frame rate at 30 frames per second.

### PRP Derived Growth Factors

Growth factors were measured by using commercially available ELISA kit. The ELISA (Enzyme-Linked Immunosorbent Assay) kit is an in vitro enzyme-linked immunosorbent assay for the quantitative measurement of samples in plasma. Exosomes were isolated by serial centrifugation steps and analyzed by Zetaview.

Data tables and descriptive statistics are shown below for each parameter (see, e.g., Tables 6 to 11).

TABLE 6
Device 1
Platelet Counts

| | Whole | PRP | | | | | | |
|---|---|---|---|---|---|---|---|---|
| | Blood | Vol. | Baseline | | | | | |
| | Vol. IN | OUT | Ct. | PRP Ct. | Fold | % | HCT | WBC |
| Donor | (ml) | (ml) | (×10e6) | (K/uL) | Increase | recovery | (%) | (k/uL) |
| 1 | 30 | 16.8 | 220 | 209 | 0.95 | 53.20 | 0 | 0.2 |
| 2 | 30 | 17.0 | 316 | 340 | 1.08 | 60.97 | 0 | 0.1 |
| 3 | 30 | 16.3 | 205 | 227 | 1.11 | 60.16 | 0 | 0.3 |
| 4 | 30 | 16.5 | 264 | 279 | 1.06 | 58.13 | 0 | 0.2 |
| 5 | 30 | 15.9 | 287 | 241 | 0.84 | 44.51 | 0.1 | 0.1 |
| Avg | | 16.5 | 258.4 | 259.2 | 1.0 | 55.4 | 0.0 | 0.2 |
| STDEV | | 0.4 | 46.1 | 52.0 | 0.1 | 6.8 | 0.0 | 0.1 |

TABLE 7
Device 1
Exosomes (Zeta View)

| | Control | Agitation |
|---|---|---|
| Donor | (1E+12) | (1E+12) |
| 1 | 1.4 | 2.0 |
| 2 | 0.9 | 1.4 |
| 3 | 1.3 | 1.7 |
| 4 | 1.5 | 1.9 |
| 5 | 1.6 | 2.2 |
| Avg | 1.3 | 1.8 |
| STDEV | 0.3 | 0.3 |

TABLE 8
Device 1
Growth Factors (ELISA)

| | VEGF | VEGF | PDGF | PDGF | FGF | FGF |
|---|---|---|---|---|---|---|
| | (pg/mL) - | (pg/mL) - | (ng/mL) - | (ng/mL) - | (pg/mL) - | (pg/mL) - |
| Donor | Control | Agitation | Control | Agitation | Control | Agitation |
| 1 | 66.3 | 99.3 | 169.1 | 244.9 | 5.3 | 6.5 |
| 2 | 48.5 | 75.4 | 218.4 | 261.5 | 5.9 | 6.7 |
| 3 | 86.3 | 122.7 | 240.2 | 303.6 | 6.1 | 7 |
| 4 | 77.9 | 118.4 | 208.7 | 290.7 | 4.7 | 5.7 |
| 5 | 63.5 | 104.5 | 188.5 | 275.2 | 5.3 | 6.2 |
| Avg | 68.5 | 104.1 | 205.0 | 275.2 | 5.5 | 6.4 |
| STDEV | 14.4 | 18.7 | 27.4 | 23.2 | 0.6 | 0.5 |

TABLE 9
Device 2
Platelet Counts

| | Whole | PRP | | | | | | |
|---|---|---|---|---|---|---|---|---|
| | Blood | Vol. | Baseline | | | | | |
| | Vol. IN | OUT | Ct. | PRP Ct. | Fold | % | HCT | WBC |
| Donor | (ml) | (ml) | (×10e6) | (K/uL) | Increase | recovery | (%) | (k/uL) |
| 1 | 38 | 10.2 | 220 | 646 | 2.94 | 78.82 | 0.4 | 1.5 |
| 2 | 38 | 9.8 | 316 | 834 | 2.64 | 68.06 | 0.3 | 2.1 |
| 3 | 38 | 10.1 | 205 | 547 | 2.67 | 70.92 | 0.2 | 1.7 |
| 4 | 38 | 10.0 | 264 | 797 | 3.02 | 79.45 | 0.2 | 2.3 |
| 5 | 38 | 10.0 | 287 | 903 | 3.15 | 82.80 | 0.2 | 2.5 |
| Avg | | 10.0 | 258.4 | 745.4 | 2.88 | 76.01 | 0.3 | 2.0 |
| STDEV | | 0.1 | 46.1 | 145.4 | 0.2 | 6.2 | 0.1 | 0.4 |

TABLE 10
Device 2
Exosomes (Zeta View)

| | Control | Agitation |
|---|---|---|
| Donor | (1E+12) | (1E+12) |
| 1 | 2.4 | 3.3 |
| 2 | 2.3 | 3.1 |
| 3 | 2.6 | 3.4 |
| 4 | 2.7 | 3.8 |
| 5 | 2.3 | 2.9 |
| Avg | 2.5 | 3.3 |
| STDEV | 0.2 | 0.3 |

TABLE 11
Device 2
Growth Factors (ELISA)

| | VEGF | VEGF | PDGF | PDGF | FGF | FGF |
|---|---|---|---|---|---|---|
| | (pg/mL) - | (pg/mL) - | (ng/mL) - | (ng/mL) - | (pg/mL) - | (pg/mL) - |
| Donor | Control | Agitation | Control | Agitation | Control | Agitation |
| 1 | 137.4 | 195.6 | 782.5 | 843.7 | 6.5 | 9.5 |
| 2 | 145.3 | 202.5 | 836.7 | 1043.5 | 7.6 | 11.5 |
| 3 | 122.8 | 187.5 | 648.3 | 776.5 | 5.8 | 8.8 |
| 4 | 141.6 | 216.4 | 876 | 1354.6 | 7.5 | 13.2 |
| 5 | 173.4 | 248.6 | 934.2 | 1557.8 | 8.8 | 14.8 |
| Avg | 144.1 | 210.1 | 815.5 | 1115.2 | 7.2 | 11.6 |
| STDEV | 18.5 | 24.0 | 108.7 | 334.2 | 1.1 | 2.5 |

As the data illustrates, in all five (5) processed samples, both Device 1 and Device 2 produced a golden straw-colored lysate with enriched platelet counts. With Device 1, platelet increase was 1.0±0.1 (X over whole blood) with platelet recovery of 55±8%. Hematocrit was below 1% in all cases. With Juva-PRP, platelet increase was 2.9±0.2 (X over whole blood) with platelet recovery of 76±6%. Hematocrit was below 1% in all cases. Results are shown in the table. Coinciding with the higher platelet counts, exosome counts were higher for Device 2 (2.5±0.2 trillion exosomes per mL) compared to Device 1 (1.3±0.3 trillion exosomes per mL). Both biological samples when exposed to 10 seconds of mechanical energy (agitation) resulted in a further, secondary increase in exosome counts. The exosomes in the sample extracted from Device 1 increased by about 40% to 1.8±0.3 trillion/mL; and the exosomes in the sample extracted from Device 2 increased 32% to 3.3±0.2 trillion/mL (see, Tables 6 to 11, below).

In addition, three growth factors were assessed, namely VEGF (pg/mL), PDGF (ng/ml) and FGF (pg/mL). Consistent with higher platelet counts and exosome counts, all three growth factors were higher in the biological sample extracted from Device 2 compared to the biological sample extracted from Device 1. (VEGF: 69±14 vs. 144±19; PDGF: 205±27 vs. 816±109; FGF 5.5±0.6 vs. 7.2±1.1). When the PRP samples were exposed to 10 seconds of mechanical energy there was a secondary increase in growth factor levels, as well. As illustrated by the data described herein, exemplary methods of the present invention provide an unexpected release of platelet-derived growth factors and exosomes.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

What is claimed is:

1. A method for:
separating and/or isolating exosomes in a post-centrifuged biological sample;
increasing the number of exosomes available for administration to a patient; and/or
enriching the exosome count in a biological sample, comprising:
centrifuging a collection tube containing a biological sample and a separation barrier (e.g., a float or a thixotropic gel); and
agitating the collection tube at an angle and rate effective to create a visually perceivable layer of foam on top of said biological sample.

2. The method according to claim 1, wherein the collection tube further comprises an anticoagulant.

3. The method according to claim 1, wherein the biological sample comprises a plurality of components.

4. The method according to claim 1, wherein the centrifugation is performed at a force of from about 500 g up to about 4000 g for a time sufficient to separate the plurality of components in the biological sample.

5. The method according to claim 1, wherein the thixotropic gel forms a barrier between the plurality of components of the biological sample.

6. The method according to claim 1, wherein the biological sample comprises whole blood.

7. The method according to claim 1, further comprising the step of activating the platelets to induce the release of exosomes.

8. The method according to claim 7, wherein the activating step comprises the application of a chemical activating agent.

9. The method according to claim 7, wherein the activating step does not involve a chemical activating agent.

10. The method according to claim 8, wherein the chemical activating agent is selected from:
calcium chloride, calcium gluconate, thrombin, collagen, lipopolysaccharide, Ca²⁺ ionophores;
and a combination of two or more thereof.

11. The method according to claim 9, wherein the activating step comprises applying energy to the biological sample.

12. The method according to claim 11, wherein the energy comprises light energy or electromagnetic energy.

13. The method according to claim 12, wherein the energy comprises infrared light.

14. The method according to claim 1, further comprising extracting at least a portion of the biological sample comprising exosomes.

15. The method according to claim 14, further comprising purifying the sample to isolate the exosomes.

16. The method according to claim 15, wherein the purification step comprises filtering the portion of the biological sample comprising exosomes.

17. A method for treating, preventing or ameliorating a symptom associated with alopecia, bed sores, wrinkles, pain, tendonitis, arthritis, acne, scarring, crow's feet, ligament sprains and tears, and/or skin lesions comprising administering a biological sample according to claim 14, to a subject in need thereof.

18. The method according to claim 17, wherein the administering step comprises application of the biological sample comprising exosomes to a skin surface of the subject in need thereof.

19. The method according to claim 18, wherein the biological sample comprising exosomes is administered via injection or via mask.
